(19) United States
(12) Patent Application Publication
(10) Pub. No.: US 2008/0112897 A1
(43) Pub. Date: May 15, 2008

(54) Product release system to atomize cosmetic hair or skin compositions containing UV filters

(76) Inventors: Hartmut Schiemann, Hunfeld (DE); Thomas Krause, Darmstadt (DE); Michael Franzke, Rossdorf (DE); Dirk Weber, Marly (CH); Monika Moenks, Schmitten (CH); Jan Baumeister, Farvagny-le-Grand (CH); Ellen Florig, Grasellenbach (DE)

Correspondence Address: THE PROCTER & GAMBLE COMPANY;INTELLECTUAL PROPERTY DIVISION - WEST BLDG., WINTON HILL BUSINESS CENTER - BOX 412, 6250 CENTER HILL AVENUE, CINCINNATI, OH, 45224, US

(21) Appl. No.: 11/471,444

(22) Filed: Jun. 20, 2006

(30) Foreign Application Priority Data
Jun. 20, 2005 (DE) 102005028388.8

(51) Int. Cl. A61K 8/37; A61K 8/36

(52) U.S. Cl. 424/47; 424/59; 424/60

(57) ABSTRACT

A product release system for atomizing cosmetic hair or skin compositions is described, which has (a) pressure-resistant packaging, (b) a capillary-containing spray head, and (c) a propellant-containing cosmetic composition containing at least one UV filter. The atomization is done using the capillary. The capillary preferably has a diameter of 0.1 to 1 mm and a length of 5 to 100 mm. The spray rate is preferably 0.01 to 5 g/s. The composition can particularly be a gel, wax, or emulsion.

The object of the present invention is a product release system to atomize cosmetic compositions having pressure-resistant packaging, a capillary-containing spray head, and a propellant-containing cosmetic composition, wherein the composition contains at least one UV filter. The object of the invention is also a corresponding method for hair or skin treatment.

Human hair and human skin can be damaged by the influence of sunlight. Particularly the so-called UVB range with wavelengths of between 290 and 320 nm as well as the so-called UVA range with wavelengths of between 320 and 400 nm are damaging. Sun-protective agents containing UV filters are used to protect skin and hair. Sun-protective agents are known in various dispensing forms, e.g., as a cream, lotion, paste, milk, or gel. To improve long-term efficacy, sun-protective agents with a high portion of oil (water-in-oil emulsions, sun oils), which do not wash off during bathing, are used. The known disadvantages of these forms of application are stickiness, a somewhat unpleasant feel on the skin for the user, and its poor distributability on the areas affected due to its oiliness and/or somewhat (creams, pastes) high viscosity. This causes an uneven layer thickness which, in turn, means insufficient protection of the skin and/or the hair. Lower-viscosity sun-protective products with easier distributability and better feel on the skin often have a lower, insufficient sun protection factor.

A process for atomizing liquid is known from WO 03/051523 A1 with which the spray is formed using a capillary. Only the application with respect to atomizing liquid compositions is described. A fixture for atomizing liquid products is described in WO 03/051522 A2, wherein the spray is formed using a capillary. Only the use of liquid compositions for atomizing, which can also be highly viscous, are described, wherein 5,000 mPa s is mentioned as the maximum sprayable viscosity.

There is a need for products that combine high sun protection factors with an improved, even, and economical applicability as well as a more pleasant feel on the skin as compared to conventional sun creams.

The object of the invention is a product release system for atomizing cosmetic compositions. The product release system has the following features:

(a) pressure-resistant packaging;

(b) a spray head containing a capillary; and

(c) a propellant-containing cosmetic composition,

wherein the atomization is done using the capillary and the composition contains at least one UV filter.

The term “atomize” is understood to mean the release of the product in the form of dissipated particles. The dissipated particles can have varying shapes, consistency, and sizes. The properties of the atomized particles can include everything from fine aerosol atomized spray to liquid drops, snow-like drops, solid spray flakes, and spray foam.

The quantities of ingredients (e.g., wt. %) indicated in the following are each based on the basic composition without propellant unless explicitly indicated otherwise. The quantities of the propellant are based on the total composition including propellant.

The composition is preferably non-liquid at 25° C. and/or has a viscosity greater than 5,000 mPa s (measured with a HAAKE VT-550 Rheometer, SV-DIN test body at a temperature of 25° C. and a shear speed of 12.9 s⁻¹). The properties of the compositions to be used according to the invention that are related to consistency are based on the base composition without propellant (unless explicitly indicated otherwise). Non-liquid compositions in terms of the invention are particularly non-flow-capable compositions, which, for example, can be determined due to the fact that they will not flow off of a glass surface tilted at 45° at a temperature of 25° C. Gel compositions are characterized in that the memory module G′ is larger than the loss module G″ at 25° C. with oscillographic measurements in the typical measurement range (0.01 to 40 Hz).

The viscosity of the composition to be used is preferably greater than 5,000 up to 100,000, or especially preferably 10,000 to 50,000 mPa s, and very especially preferably 25,000 to 35,000 mPa s, measured with a HAAKE VT-550 Rheometer, SV-DIN test body at a temperature of 25° C. and a shear speed of 12.9 s⁻¹.

Aerosol spray cans constructed of metal or plastic can be used as the pressure-resistant packaging. Preferred metals are tin plates and aluminum, while the preferred plastic is polyethylene terephthalate.

Suitable spray systems with capillary-containing spray heads, with which the spray is formed using a capillary, are described in WO 03/051523 A1 and in WO 03/051522 A2. The capillaries preferably have a diameter of 0.1 to 1 mm, or particularly of 0.2 to 0.6 mm and a length that is preferably 5 to 100 mm, or particularly 5 to 50 mm. The spray principle is also described in Aerosol Europe, vol. 13, no. 1-2005, pages 6-11. The spray system is based on the principle of capillary atomization. The conventional swirl nozzle as well as, if necessary, the uptake tube are replaced by capillaries. The energy-consuming and propellant-intensive swirling of the content of the can and the required strong dilution of the product with solvents is not necessary as compared to conventional spray systems. Even if only a small quantity of propellant is used, the product rises upward on the wall of the uptake tube capillary and is propelled, after the valve in the (wider) capillary of the spray head, in the direction of the exit opening. In this manner, small drops from the flowing propellant are torn from the surface of the liquid and continue to flow as aerosol. Since there is no swirl chamber to inhibit the flow of the product nor any atomizing nozzle available, the energy in the system can be used much more efficiently to create the desired spray. The spray rate can be adjusted via the selection of the capillary geometry in conjunction with the interior pressure created by the propellant or a propellant mixture. Preferred spray rates are 0.01 to 0.5 g/s, or particularly 0.1 to 0.3 g/s. The size of the spray drops created with the atomization can be adjusted via the selection of the capillary geometry in conjunction with the interior pressure or the viscosity of the composition. Suitable capillary atomization systems can be obtained in a product called TRUSPRAY® from Boehringer Ingelheim microParts GmbH.

The preferred drop size distributions are those with which the dv(50) value is a maximum of 200 μm, e.g., of from 50 to 200 μm, with a maximum of 100 μm being especially preferred, e.g., of from 70 to 90 μm and/or with which the dv(90) value is a maximum of from 160 μm, e.g., of from 90 to 160 μm, with a maximum of 150 μm being especially preferred, e.g., of from 115 to 150 μm. The dv(50) or dv(90) values indicate the maximum diameter that 50% or 90% of all droplets have. The drop size distribution can, for example, be determined with the help of a particle measurement unit based on laser beam diffraction, e.g., a Malvern particle sizer measuring device. Compositions that form a snow-like consistency, flakes, or foam (spray foam) upon exiting the capillary spray system are also preferred.

The propellant to be used can be selected from lower alkanes, particularly C3 to C5 hydrocarbons such as, for example, n-butane, i-butane, and propane, or also mixtures thereof, as well as dimethylethers or fluorine hydrocarbons such as F 152a (1,1-difluoroethane) or F 134 (tetrafluoroethane) as well as other gaseous propellants present with the pressures considered, such as, for example, N₂, N₂O, and CO₂ as well as mixtures of the aforementioned propellants. The propellant is preferably selected from propane, n-butane, isobutane, dimethylether, fluorinated hydrocarbons, and mixtures thereof. The content of propellant is, in addition, preferably 15 to 85 wt. %, with 25 to 75 wt. % being especially preferred.

The composition contains cosmetically acceptable solvents, preferably an aqueous, alcoholic, or aqueous alcoholic medium. The lower alcohols with 1 to 4 C atoms, such as ethanol and isopropanol, can be contained as alcohols, particularly those typically used for cosmetic purposes. The composition can be in a pH range of 2.0 to 9.5. A pH range of 4 to 8 is particular preferred, providing no special application forms require other pH values. As additional co-solvents, organic solvents or a mixture of solvents with a boiling point of less than 400° C. can be contained in a quantity of from 0.1 to 15 wt. % or preferably of from to 10 wt. %. Unbranched or branched hydrocarbons such as n-pentane, hexane, isopentane, and cyclic hydrocarbons such as cyclopentane and cyclohexane are particularly suitable as additional co-solvents. These volatile hydrocarbons can also be used as propellants. Other, especially preferred water-soluble solvents are glycerin, ethylene glycol, and propylene glycol in a quantity of up to 30 wt. %.

The product release system according to the invention can be used for hair and/or skin treatment. The compositions can be agents for the care of hair such as, for example, hair-repair products or hair rinses, which, for example, can be applied as leave-on or rinse-off products; agents for the temporary reshaping and/or stabilizing of the hairstyle (styling agent), for example hair sprays, hair lacquers, hair gels, hair waxes, styling creams, etc.; permanent, semipermanent, or temporary hair colorants, for example oxidative hair colorants or nonoxidative hair tinting agents or hair bleaching agents; permanent hair restructuring agents, for example in the form of a mildly alkaline or acidic permanent wave or hair straightening agents containing a reducing agent, or in the form of permanent wave fixing agents containing an oxidizing agent. Due to the content of UV filters, the hair treatment products can be equipped with an additional photoprotective effect for hair.

The UV filters are contained in the composition to be used according to the invention preferably in a quantity of from 0.01 to 40 wt. %, particularly of from 0.1 to 30 wt. %, or of from 0.5 to 20 wt. %, or of from 1 to 15 wt. %, in relation to the total weight of the composition without propellant in each case.

UV filters are understood to mean those types of materials that reduce UV radiation via one or more effects after being applied to the skin or the hair. The effects can be, for example, reflection or dispersion of the radiation on the surface of powdery solid materials (physical photo-protection) or absorption of chemical substances (chemical photo-protection). Depending on which wavelength range is being absorbed, a distinction is made between UVB filters (absorption range 280 bis 320 nm), UVA filters (absorption range 320 bis 400 nm), and broadband filters (absorption range 290 to approx. 380 nm).

Suitable UV filters are, for example, derivatives of 3-benzylidene camphor, 4-aminobenzoic acid, cinnamic acid, salicylic acid, benzophenone, 2-phenylbenzimidazole; dibenzoylmethane derivatives, in particular:
Water-soluble, sulfonated UVA filter substances such as, for example, phenylene-1,4-bis-(2-benzimidazyl)-3,3′-5,5′-tetrasulfonic acid and salts thereof, particularly bis-sodium salt;
UVB/UVA filters such as, for example, asymmetrically substituted triazine compounds such as, for example, 2,4-bis-{[4-(2-ethylhexyloxy)-2-hydroxy]phenyl}-6-(4-methoxyphenyl)-1,3,5-triazine (INCI: Bis-Ethylhexyloxylphenol Methoxyphenyl Triazine), certain benzophenones such as, for example, 2-hydroxy-4-methoxy benzophenone (INCI: Benzophenone-3) or 2,2′-methylene-bis-(6-(2H-benzotriazol-2-yl)-4-(1,1,3,3-tetramethylbutyl)phenol) (INCI: Methylene Bis-Benzotriazolyl Tetramethylenebutylphenol);
UV filter substances that are liquid at room temperature, for example, homomenthyl salicylate (INCI: Homosalate), 2-ethylhexyl-2-cyano-3,3-diphenyl acrylate (INCI: Octocrylene), 2-ethylhexyl-2-hydroxybenzoate (2-ethylhexylsalicylate, octylsalicylate, INCI: Ethylhexyl Salicylate); and esters of cinnamic acid, preferably 4-methoxycinnamic acid(2-ethylhexyl)ester (2-ethylhexyl-4-methoxycinnamate, INCI: Ethylhexyl Methoxycinnamate), and 4-methoxycinnamic acid isopentyl ester (Isopentyl-4-methoxycinnamat, INCI: Isoamyl p-Methoxycinnamate), 3-(4-(2,2-bis ethoxycarbonylvinyl)phenoxy)propenyl)methoxysiloxane/dimethylsiloxane copolymer;
Inorganic pigments, particularly metallic oxides and/or other metallic compounds that are difficult to dissolve or are insoluble in water, particularly oxides of titanium (TiO₂), zinc (ZnO), iron (e. g., Fe₂O₃), zirconium (ZrO₂), silicon (SiO₂), manganese (e. g., MnO), aluminum (Al₂O₃), cerium (e. g., Ce₂O₃), mixed oxides of the corresponding metals as well as mixtures of oxides such as these, and the sulfate of barium (BaSO₄);
The pigments can be used in the form of commercially obtainable oily or aqueous precursor dispersions. Dispersion aids and/or solubilization facilitators can be added to these precursor dispersions. Pigments can be surface-treated (coated), wherein, for example, a hydrophilic, amphipathic, or hydrophobic character can be formed or should be retained. The surface treatment can exist in that the pigments are provided with a thin, hydrophilic and/or hydrophobic inorganic and/or organic layer according to a known method. The various surface coatings can also contain water. Inorganic surface coatings can consist of aluminum oxide (Al₂O₃), aluminum hydroxide (Al(OH)₃), or aluminum oxide hydrate (also: alumina, CAS no.: 1333-84-2), sodium hexametaphosphate ((NaPO₃)₆), sodium metaphosphate ((NaPO₃)ₙ), silicium dioxide (SiO₂) (also: silica, CAS no.: 7631-86-9), or iron oxide (Fe₂O₃). These inorganic surface coatings can be present alone, in a combination, and/or in conjunction with organic coating materials. Organic coating materials can consist of aluminum stearate, stearic acid, lauric acid, dimethylpolysiloxane (also: dimethicone), methylpolysiloxane (methicone), simethicone (a mixture of dimethylpolysiloxane with an average chain length of 200 to 350 dimethylsiloxane units and silica gel), or alginic acid. These inorganic surface coatings can be present alone, in a combination, and/or in conjunction with inorganic coating materials.
Additional advantageous pigments are latex particles, e.g., as described in U.S. Pat. No. 5,663,213 and EP 0 761 201. Particularly advantageous latex particles are those that are formed from water and styrene/acrylate copolymers and, for example, that can be obtained under the trade name “Alliance SunSphere” from Rohm & Haas.
Dibenzoylmethane derivatives, particularly 4-(tert.-butyl)-4′-methoxydibenzoylmethane (CAS no.: 70356-09-1; Parsol® 1789; Eusolex® 9020);
Sulfonated, water-soluble UV filters such as:
   Phenylene-1,4-bis-(2-benzimidazyl)-3,3′-5,5′-tetrasulfonic acid and salts thereof, particularly the corresponding sodium, potassium, or triethanol ammonium salts, particularly phenylene-1,4-bis-(2-benzimidazyl)-3,3′-5,5′-tetrasulfonic acid-bis-sodium salt with the INCI designation Disodium Phenyl Dibenzimidazole Tetrasulfonate (CAS no.: 180898-37-7, Neo Heliopan AP);
   Salts of 2-phenylbenzimidazole-5-sulfonic acid, such as its sodium, potassium, or triethanol ammonium salt, as well as the sulfonic acid itself with the INCI designation Phenylbenzimidazole Sulfonic acid (CAS. no.: 27503-81-7; Eusolex 232; Neo Heliopan Hydro);
   1,4-di(2-oxo-10-sulfo-3-bornylidenmethyl)benzene (also: 3,3′-(1,4-phenylendimethylene)-bis-(7,7-dimethyl-2-oxo-bicyclo-[22.1]hept-1-ylmethane sulfonic acid) and its salts, particularly the sodium, potassium, or triethanol ammonium salt; also characterized as benzene-1,4-di(2-oxo-3-bornylidenmethyl-10-sulfonic acid) (INCI: Terephthalylidene Dicamphor Sulfonic acid; CAS. no.: 90457-82-2; Mexoryl SX);
   Sulfonic acid derivatives of 3-benzylidene camphor such as, for example, 4-(2-oxo-3-bornylidenmethyl)benzene sulfonic acid, 2-methyl-5-(2-oxo-3-bornylidenmethyl)sulfonic acid, and salts thereof,
   Benzoxazole derivatives, particularly those of the formula

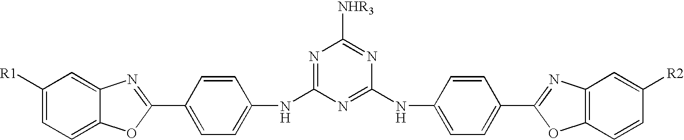

wherein R¹, R², and R³, independently from one another, are selected from the group of branched or unbranched, saturated or unsaturated alkyl groups with 1 to 10 C atoms. It is particularly advantageous according to the invention if the radicals R¹ and R² are selected to be equal, particularly from the group of branched alkyl groups with 3 to 5 C atoms. It is further particularly advantageous if R³ represents an unbranched or branched alkyl group with 8 C atoms, particularly the 2-ethylhexyl radical. A preferred benzoxazole derivative is 2,4-bis-[5-1 (dimethylpropyl)benzoxazole-2-yl-(4-phenyl)imino]-6-(2-ethylhexyl)imino-1,3,5-triazine with CAS no. 288254-16-0 (Uvasorb® K2A). The benzoxazole derivative(s) is (are) advantageously present in dissolved form in the cosmetic preparations according to the invention. However, it can, if necessary, also be advantageous if the benzoxazole derivative(s) is (are) present as a pigment, i.e., in an undissolved form—for example in particle sizes of 10 nm up to 300 nm.
Hydroxybenzophenones, particularly those of the formula

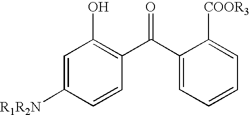

wherein R¹ and R², independently from one another, mean hydrogen, C₁-C₂₀ alkyl, C₃-C₁₀ cycloalkyl, or C₃-C₁₀ cycloalkenyl, wherein the substituents R¹ and R², along with the nitrogen atom to which they are bound, can form a 5 or 6 ring, and R³ means a C₁-C₂₀ alkyl radical. A particularly advantageous hydroxybenzophenone is 2-(4′-diethylamino-2′-hydoxybenzoyl)benzoic acid hexyl ester (also: aminobenzophenone; Uvinul® A Plus).
Triazine derivatives such as, for example, 2,4-bis-{[4-(2-ethylhexyloxy)-2-hydroxy]phenyl}-6-(4-methoxyphenyl)-1,3,5-triazine (INCI: Bis-Ethylhexyloxylphenol Methoxyphenyl Triazine; Tinosorb® S); dioctylbutylamidotriazone (INCI: Diethylhexyl Butamido Triazone; UVASORB HEB); 4,4′,4″-(1,3,5-triazine-2,4,6-triyltriimino)trisbenzoic acid tris(2-ethylhexyl ester), also: 2,4,6-tris-[anilino-(p-carbo-2′-ethyl-1′-hexyloxy)]-1,3,5-triazine (INCI: Ethylhexyl Triazone; UVINUL® T 150); 2-[4,6-bis(2,4-dimethylphenyl)-1,3,5-triazin-2-yl]-5-(octyloxy)phenol (CAS no.: 2725-22-6), 2,4,6-trianilino-(p-carbo-2′-ethyl-1′-hexyloxy)-1,3,5-triazine;
2,2′-methylen-bis-(6-(2H-benzotriazol-2-yl)-4-(1,1,3,3-tetramethylbutyl)phenol) (INCI: Methylene Bis-Benztriazolyl Tetramethylbutylphenol; Tinosorb® M);
2-(2H-benzotriazol-2-yl)-4-methyl-6-[2-methyl-3-[1,3,3,3-tetramethyl-1-[(trimethylsilyl)oxy]disiloxanyl]propyl]phenol (CAS no.: 155633-54-8; INCI: Drometrizole Trisiloxane);
Benzylidene camphor derivatives, preferably 3-(4-methylbenzylidene)camphor, 3-benzylidene camphor, 4-methylbenzylidene camphor (Eusolex® 6300);
4-Aminobenzoic acid derivatives, preferably 4-(dimethylamino)benzoic acid(2-ethylhexyl)ester, 4-(dimethylamino)benzoic acid amyl ester;
Esters of benzalmalonic acid, preferably 4-methoxybenzalmalonic acid di(2-ethylhexyl)ester;
Esters of cinnamic acid, preferably 4-methoxycinnamic acid(2-ethylhexyl)ester, 4-methoxycinnamic acid isopentyl ester;
Derivatives of benzophenone, preferably 2-hydroxy-4-methoxybenzophenone, 2-hydroxy-4-methoxy-4′-methylbenzophenone, 2,2′-dihydroxy-4-methoxybenzophenone;
Sulfonic acid derivatives of 3-benzylidene camphor such as, for example, 4-(2-oxo-3-bornylidene methyl)benzene sulfonic acid, 2-methyl-5-(2-oxo-3-bornylidene methyl)sulfonic acid, and salts thereof;
Ethylhexyl-2-cyano-3,3-diphenyl acrylate (Octocrylen, Uvinul® N 539 T);
As well as UV filters bound to polymers.

Preferred sulfonic-acid-substituted benzimidazoles and benzoxazoles have, for example, the general formula

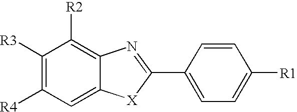

wherein R1 stands for hydrogen, a C1 to C10 alkoxy group, or for the group;

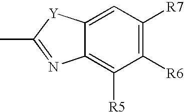

R2 to R7, independently from one another, stand for hydrogen and for —SO₃H, wherein at least one of the radicals R2 to R7 stands for —SO₃H;
X and Y stand for oxygen or NH.

Especially preferred are compounds with which X and Y (if available) equal NH and wherein R2 and R4 as well as R5 and R7 are all equal to and different from R3 and/or R6.

Benzimidazoles are, for example, 2-phenyl-1H-benzimidazole-5-sulfonic acid (e.g., Neo Heliopan® Hydro), 2,2′-(1,4-phenylene)bis-(1H-benzimidazole-4,6-disulfonic acid) (e.g., Neo Heliopan® AP), and 2,2′-(1,4-phenylene)bis-(1H-benzimidazole-5-sulfonic acid) and their particular salts. A benzoxazole is, for example, 2,2′-(1,4-phenylene)bis-(oxazole-5-sulfonic acid) and its salts.

Especially preferred are 4-tert-butyl-4′-methoxydibenzoylmethane; 4-methoxycinnamic acid octyl ester, particularly 4-methoxycinnamic acid-2-ethylhexyl ester; methyl methoxy cinnamate; 2-phenyl-5-sulfobenzimidazole (INCI: Phenylbenzimidazole Sulfonic Acid); zinc oxide; titanium dioxide; ethylhexyl triazone; 2-hydroxy-4-methoxybenzophenone-5-sulfonic acid; 3-(4-methylbenzylidene)-dl-camphor (INCI: 4-Methylbenzylidene Camphor); polyethoxylated p-aminobenzoates; and 2-ethylhexyl-2-cyano-3,3-diphenyl acrylate (INCI: Octocrylene).

In one embodiment, the composition to be used according to the invention is gel-like and contains at least one thickener or gel-former preferably in a quantity of from 0.01 to 20 wt. % or 0.05 to 10 wt. % or especially preferably of from 0.1 to 5 wt. %. Materials for which the function “Viscosity Increasing Agent” is indicated in the International Cosmetic Ingredient Dictionary and Handbook, 10th edition, 2004 are essentially suitable. The thickener or gel-former is preferably a thickening polymer and is especially preferably selected from copolymers consisting of at least one first type of monomer, which is selected from acrylic acid and methacrylic acid, and at least one second type of monomer, which is selected from esters of acrylic acid and ethoxylated fatty alcohol; crosslinked polyacrylic acid; crosslinked copolymers consisting of at least one first type of monomer, which is selected from acrylic acid and methacrylic acid, and at least one second type of monomer, which is selected from esters of acrylic acid with C10 to C30 alcohols; copolymers consisting of at least one first type of monomer, which is selected from acrylic acid and methacrylic acid, and at least one second type of monomer, which is selected from esters of itaconic acid and ethoxylated fatty alcohol; copolymers consisting of at least one type of monomer, which is selected from acrylic acid and methacrylic acid, at least one second type of monomer, which is selected from esters of itaconic acid and ethoxylated C10 to C30 alcohol, and a third type of monomer, which is selected from C1 to C4 aminoalkyl acrylates; copolymers consisting of two or more monomers, which are selected from acrylic acid, methacrylic acid, acrylic acid esters and methacrylic acid esters; copolymers consisting of vinyl pyrrolidone and ammonium acryloyl dimethyltaurate; copolymers consisting of ammonium acryloyl dimethyltaurate and monomers selected from esters of methacrylic acid and ethoxylated fatty alcohols; hydroxyethyl cellulose; hydroxypropyl cellulose; hydroxypropyl guar; glyceryl polyacrylate; glyceryl polymethacrylate; copolymers consisting of at least one C2, C3, or C4 alkylene and styrene; polyurethane; hydroxypropyl starch phosphate; polyacrylamide; copolymers crosslinked with decadiene consisting of maleic acid anhydride and methyl vinyl ether; locust bean gum; guar gum; xanthan; dehydroxanthan; carrageenan; karaya gum; hydrolyzed corn starch; copolymers consisting of polyethylene oxide, fatty alcohols, and saturated methylene diphenyl diisocyanate (e.g., PEG-150/stearyl alcohol/SMDI copolymer).

In an additional embodiment, the composition is waxy and contains at least one wax that is solid at 25° C. in a quantity of preferably from 10 to 80 wt. %, particularly of from 20 to 60 wt. %, or of from 25 to 50 wt. %, as well as, if necessary, other water-insoluble materials that are liquid at room temperature. The waxy consistency is preferably characterized in that the needle penetration number (unit of measurement 0.1 mm), test weight 100 g, testing time 5 s, test temperature 25° C. (according to DIN 51 579) preferably ranges from 2 to 70, or particularly from 3 to 40, and/or that the composition can be melted and has a solidification point that is greater than 25° C., or is preferably in a range of from 30 to 70° C., or especially preferably in a range of from 40 to 55° C.

Principally any wax that is known in the prior art can be used as a wax or waxy material. These waxes include animal, vegetable, mineral, and synthetic waxes, microcrystalline waxes, macrocrystalline waxes, solid paraffins, petroleum jelly, Vaseline, ozocerite, montan wax, Fischer-Tropsch wax, polyolefin waxes, e.g., polybutene, beeswax, wool wax, and its derivatives such as, for example, wool wax alcohols, candelilla wax, olive wax, carnauba wax, Japan wax, apple wax, hydrogenated fats, fatty acid esters, fatty acid glycerides with a solidification point greater than 40° C., silicone waxes or hydrophilic waxes such as, for example, high-molecular-weight polyethylene glycol waxes with a molecular weight of from 800 to 20,000, preferably of from 2,000 to 10,000 g/mol. The waxes or waxy materials have a solidification point greater than 25° C., or preferably greater than 40° C. or 55° C. The needle penetration number 0.1 mm, 100 g, 5 s, 25° C. (according to DIN 51 579) preferably lies in the range of from 2 to 70, or especially 3 to 40.

In another embodiment, the composition is emulsion-like, wherein the consistency is preferably creamy. The emulsion can be a water-in-oil emulsion, an oil-in-water emulsion, a microemulsion, or a higher emulsion. In addition to water, preferably at least one hydrophobic oil that is liquid at room temperature (25° C.) as well as at least one emulsifier is contained. The oil content is preferably 1 to 20 wt. %, or particularly 2 to 10 wt. %. The emulsifier content is preferably 0.01 to 30 wt. %, and particularly 0.1 to 20 wt. % or 0.5 to 10 wt. %.

Suitable emulsifiers can include nonionic, anionic, cationic, or zwitterionic surfactants. Suitable nonionic surfactants are, for example,
ethoxylated fatty alcohols, fatty acids, fatty acid glycerides, or alkyl phenols, especially addition products of 2 to 30 mol ethylene oxide and/or 1 to 5 mol propylene oxide to C8 to C22 fatty alcohols, to C12 to C22 fatty acids, or to alkyl phenols with 8 to 15 C atoms in the alkyl group;
C12 to C22 fatty acid mono- and diesters of addition products of 1 to 30 mol ethylene oxide to glycerol;
addition products of 5 to 60 mol ethylene oxide to castor oil or hydrogenated castor oil;
fatty acid sugar esters, especially esters from saccharose and one or two C8 to C22 fatty acids, INCI: Sucrose Cocoate, Sucrose Dilaurate, Sucrose Distearate, Sucrose Laurate, Sucrose Myristate, Sucrose Oleate, Sucrose Palmitate, Sucrose Ricinoleate, Sucrose Stearate;
esters from sorbitan and one, two or three C8 to C22 fatty acids and a degree of ethoxylation of 4 to 20;
polyglyceryl fatty acid esters, especially from one, two or more C8 to C22 fatty acids and polyglycerol with preferably 2 to 20 glyceryl units;
alkylglucosides, alkyloligoglucosides, and alkylpolyglucoside with C8 to C22 alkyl groups, e.g. decyl glucoside or lauryl glucoside.

Suitable anionic surfactants are, for example, salts and esters of carboxylic acids, alkyl ether sulfates and alkyl sulfates, fatty alcohol ether sulfates, sulfonic acids and their salts (e.g., sulfosuccinates or fatty acid isethienates), phosphoric acid esters and their salts, acylamino acids and their salts. A comprehensive description of these anionic surfactants is found in the publication “FIEDLER—Lexikon der Hilfsstoffe”[FIEDLER—Dictionary of Adjuvants], volume 1, fifth edition (2002), pages 97 to 102, to which expressed reference is made. Preferred surfactants are mono-, di-, and/or triesters of phosphoric acid with addition products of from 2 to 30 mol ethylene oxide to C8 to C22 fatty alcohols.

Suitable amphoteric surfactants are, for example, derivatives of aliphatic quaternary ammonium, phosphonium and sulfonium compounds of the formula

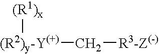

wherein R¹ represents a straight-chain or branched-chain alkyl, alkenyl, or hydroxyalkyl group with 8 to 18 C atoms and 0 to about 10 ethylene oxide units and 0 to 1 glycerol units; Y is an N-, P-, or S-containing group; R² is an alkyl or monohydroxyalkyl group with 1 to 3 C atoms; the total of x+y equals 2 if Y is a sulfur atom, and the total of x+y equals 3 if Y is a nitrogen atom or a phosphorus atom; R³ is an alkylene or hydroxyalkylene group with 1 to 4 C atoms, and Z(−) represents a carboxylate, sulfate, phosphonate, or phosphate group.

Other amphoteric surfactants such as betaines are also suitable. Examples of betaines include C8 to C18 alkylbetaines such as cocodimethylcarboxymethylbetaine, lauryldimethylcarboxymethylbetaine, lauryldimethyl-alpha-carboxyethylbetaine, cetyldimethylcarboxymethylbetaine, oleyldimethylgammacarboxypropylbetaine, and lauryl-bis-(2-hydroxypropyl)-alpha-carboxyethylbetaine; C8 to C18 sulfobetaines such as cocodimethylsulfopropylbetaine, stearyldimethylsulfopropylbetaine, lauryldimethylsulfoethylbetaine, lauryl-bis-(2-hydroxyethyl)sulfopropylbetaine; the carboxyl derivatives of imidazole, C8 to C18 alkyldimethylammonium acetate, C8 to C18 alkyldimethylcarbonylmethylammonium salts, as well as C8 to C18 fatty acid alkylamidobetaines such as, for example, coconut fatty acid amidopropylbetaine and N-coconut fatty acid amidoethyl-N-[2-(carboxymethoxy)ethyl]-glycerin (CTFA name: cocoamphocarboxyglycinate).

The cosmetic composition to be used according to the present invention can also contain at least one additional active cosmetic ingredient or additive for the hair or skin/scalp. This active ingredient or additive can, for example, be selected from hair-conditioning materials, hair-setting materials, photo-protection materials, preservatives, pigments, direct-penetrating hair dyes, particle-shaped materials, oxidizing agents, reducing agents, and oxidative hair colorant precursor products. Skin-care active ingredients and additives are, for example, antioxidants, natural ingredients and their derivatives, anti-wrinkle ingredients, self-tanning substances, repellents, moisturizers, and fillers to improve the feel of the skin. The active ingredients and additives, depending on the type and intended use, are preferably contained in a quantity of from 0.001 to 30 wt. %, or particularly of from 0.05 to 10 wt. %, or of from 0.1 to 5 wt. %.

The quantity of antioxidants (one or more) is preferably 0.001 to 30 wt. %, especially preferably 0.05 to 20 wt. %, or particularly 0.1 to 5 wt. %, in relation to the total weight of the preparation without propellant. Antioxidants are, for example, amino acids (e.g., glycine, histidine, tyrosine, tryptophan) and derivatives thereof, imidazoles (e.g., urocanic acid) and derivatives thereof, peptides such as D-carnosine, L-carnosine, and derivatives thereof (e.g., anserine), chlorogenic acid and derivatives thereof, aurothioglucose, propylthiouracil and other thiols (e.g., thioredoxin, glutathione, cysteine, cystine, cystamine and its glycosyl, N-acetyl, methyl, ethyl, propyl, amyl, butyl, as well as lauryl, palmitoyl, oleyl, γ-linoleyl, cholesteryl, and glyceryl esters), as well as salts thereof, dilauryl thiodipropionate, distearyl thiodipropionate, thiodipropionic acid, and derivatives thereof (esters, ethers, peptides, lipids, nucleotides, nucleosides, and salts), as well as sulfoximine compounds (e.g., buthionine sulfoximines, homocysteine sulfoximine, buthionine sulfones, penta, hexa, and heptathionine sulfoximine) in very small compatible dosages (e.g., pmol up to μmol/kg), as well as (metallic) chelating agents (e.g., α-hydroxy fatty acids, palmitic acid, phytic acid, lactoferrin), α-hydroxy acids (e.g., citric acid, lactic acid, malic acid), humic acid, gallic acid, gallic extracts, bilirubin, biliverdin, unsaturated fatty acids, and derivatives thereof (e.g., γ-linolenic acid, linoleic acid, oleic acid), folic acid and derivatives thereof, as well as coniferyl benzoate of benzoic resin, rutinic acid, and derivatives thereof, propyl gallate, ferulic acid, furfurylidene glucitol, butyl hydroxy toluene, butyl hydroxy anisole, nordihydroguaiac resin acid, nordihydroguaiaretic acid, trihydroxybutyrophenone, uric acid, and derivatives thereof, mannose and derivatives thereof, zinc and derivatives thereof (e.g., ZnO, ZnSO₄) selenium and derivatives thereof (e.g., selenium methionine), stilbenes and derivatives thereof (e.g., stilbene oxide, trans-stilbene oxide), and the derivatives of the ingredients mentioned that suitable according to the invention (salts, esters, ethers, sugars, nucleotides, nucleosides, peptides, and lipids); water-soluable antioxidants such as, for example, vitamins, e.g., ascorbic acid and derivatives thereof. Preferred antioxidants also include vitamin E (α-tocopherol) and derivatives thereof as well as vitamin A (retinol) and derivatives thereof.

Natural skin ingredients and derivatives thereof are, for example, alpha-lipoic acid and derivatives thereof, phytoen, D-biotin, coenzyme Q10 (ubiquinone and/or ubiquinol and derivatives thereof), alpha-glucosylrutin, carnitine, carnosine, natural or synthetic isoflavonoids, creatine, taurine, β-alanine, as well as 8-hexadecene-1,16-dicarboxylic acid (dioic acid). Anti-wrinkle ingredients are, for example, flavonglycosides (particularly α-glycosylrutin), coenzyme Q10, vitamin E, and derivatives thereof. Self-tanning substances are, for example, dihydroxyacetone, erythrulose, and melanine derivatives. Repellents to protect against mosquitos, ticks, and spiders and the like are, for example, N,N-diethyl-3-methylbenzamide (metadelphene, “DEET”), dimethyl phthalate (palatinol M, DMP), as well as, in particular, 3-(N-n-butyl-N-acetyl amino)propionic acid ethyl ester (Insect Repellent IR3535®). The repellents can be used individually or in a combination. The fillers to improve the feel of the skin are, for example, starches and starch derivatives (e. g., tapioca starch, distarch phosphate, aluminum or sodium starch octenylsuccinate), pigments having neither UV filters nor a coloring effect (e. g., boron nitride), and/or Aerosile® (CAS no. 7631-86-9). Moisturizers are materials or material mixtures that give cosmetic or dermatological preparations the property of reducing the moisture loss of the horny layer (transepidermal water loss, TEWL) and/or positively influencing hydration of the horny layer, after they are distributed to the skin's surface. Moisturizers are, for example, glycerin, lactic acid, and/or lactates, particularly sodium lactate, butylene glycol, propylene glycol, panthenol, dipropylene glycol, sorbitol, biosaccharide gum-1, glycine soya, ethylhexyloxy glycerine, pyrrolidone carboxylic acid, urea, polymer moisturizers from the group of water-soluble polysaccharides or polysaccharides that swell or form a gel in water, for example, hyaluronic acid, chitosan, or fucose-rich polysaccharides (Fucogel® 1000).

In one embodiment, the agent according to the invention, as a hair-care or hair-setting additive, contains at least one polymer with anionic groups or groups that can be anionized preferably in a quantity of from 0.01 to 20 wt. % or of from 0.05 to 10 wt. %, with 0.1 to 5 wt. % being particularly preferred. Groups that can be anionized are understood to be acid groups such as, for example, carboxylic acid, sulfonic acid, or phosphoric acid groups that can be deprotonated using typical bases such as, for example, organic amines or alkali- or alkaline earth hydroxides. The anionic polymers can be partially or completely neutralized with an alkaline neutralizing agent. Such types of agents in which the acidic groups are neutralized in the polymer to 50 to 100%, or especially preferably to 70-100%, are preferred. Organic or inorganic bases can be used as the neutralizing agent. Particular examples of bases are amino alkanols such as, for example, aminomethylpropanol (AMP), triethanolamine or monoethanolamine, and also ammonia, NaOH, and KOH among others.

The anionic polymer can be a homo- or copolymer with acid group-containing monomer units derived from natural or synthetic sources, which, if necessary, can be polymerized with comonomers that contain no acid groups. Among the acid groups that can be considered are sulfonic acid, phosphoric acid, and carboxylic acid groups, of which the carboxylic acid groups are preferred. Suitable acid group-containing monomers are, for example, acrylic acid, methacrylic acid, crotonic acid, maleic acid, and maleic anhydride, maleic acid monoesters, especially the C1 to C7 alkyl monoesters of maleic acid, as well as aldehydocarboxylic acids or ketocarboxylic acids. Comonomers that are not substituted with acid groups are, for example, acrylamide, methacrylamide, alkyl- and dialkylacrylamide, alkyl and dialkylmethacrylamide, alkyl acrylate, alkyl methacrylate, vinylcaprolactone, vinylpyrrolidone, vinyl ester, vinyl alcohol, propylene glycol or ethylene glycol, amine-substituted vinyl monomers such as, for example, dialkylaminoalkyl acrylate, dialkylaminoalkyl methacrylate, monoalkylaminoalkyl acrylate, and monoalkylaminoalkyl methacrylate, wherein the alkyl groups of these monomers are preferably C1 to C7 alkyl groups, with C1 to C3 alkyl groups being especially preferred.

Suitable polymers with acid groups are especially homopolymers of acrylic acid or methacrylic acid, copolymers of acrylic acid or methacrylic acid with monomers selected from acrylic acid or methacrylic acid esters, acrylamides, methacrylamides and vinylpyrrolidone, homopolymers of crotonic acid as well as copolymers of crotonic acid with monomers selected from vinyl esters, acrylic acid or methacrylic acid esters, acrylamides and methacrylamides that are uncrosslinked or crosslinked with polyfunctional agents. A suitable natural polymer is, for example, shellac.

Preferred polymers with acid groups are:

Terpolymers from acrylic acid, alkyl acrylate, and N-alkylacrylamide (INCI designation: Acrylate/Acrylamide Copolymer), especially terpolymers from acrylic acid, ethyl acrylate and N-tert-butylacrylamide; crosslinked or uncrosslinked vinyl acetate/crotonic acid copolymers (INCI designation: VA/Crotonate Copolymer); copolymers from one or more C1 to C5 alkyl acrylates, especially C2 to C4 alkyl acrylates and at least one monomer selected from acrylic acid or methacrylic acid (INCI designation: Acrylate Copolymer), e.g., terpolymers from tert-butyl acrylate, ethyl acrylate and methacrylic acid; sodium polystyrenesulfonate; vinylacetate/crotonic acid/vinyl alkanoate copolymers, for example, copolymers from vinyl acetate, crotonic acid and vinyl propionate; copolymers from vinyl acetate, crotonic acid and vinyl neodecanoate (INCI designations: VA/Crotonate/Vinyl Propionate Copolymer, VA/Crotonate/Vinyl Neodecanoate Copolymer); aminomethylpropanol acrylate copolymers; copolymers from vinylpyrrolidone and at least one further monomer selected from acrylic acid and methacrylic acid as well as, if necessary, acrylic acid esters and methacrylic acid esters; copolymers from methyl vinyl ether and maleic acid monoalkylesters (INCI designations: Ethyl Ester of PVM/MA Copolymer, Butyl Ester of PVM/MA Copolymer); aminomethylpropanol salts of copolymers from allyl methacrylate and at least one further monomer selected from acrylic acid, and methacrylic acid as well as, if necessary, acrylic acid esters and methacrylic acid esters; crosslinked copolymers from ethyl acrylate and methacrylic acid; copolymers from vinyl acetate, mono-n-butyl maleate and isobornyl acrylate; copolymers from two or more monomers selected from acrylic acid and methacrylic acid as well as, if necessary, acrylic acid esters and methacrylic acid esters; copolymers from octylacrylamide and at least one monomer selected from acrylic acid and methacrylic acid as well as, if necessary, acrylic acid esters and methacrylic acid esters; polyesters from diglycol, cyclohexanedimethanol, isophthalic acid and sulfoisophthalic acid, wherein the alkyl groups of the aforementioned polymers as a rule preferably possess 1, 2, 3, or 4 C atoms.

In one embodiment, the agent according to the invention, as a hair-conditioning or hair-setting additive, contains at least one zwitterionic and/or amphoteric polymer preferably in a quantity of from 0.01 to 20 wt. % or of from 0.05 to 10 wt. %, or especially preferably of from 0.1 to 5 wt. %. Zwitterionic polymers simultaneously have at least one anionic and at least one cationic charge. Amphoteric polymers exhibit at least one acidic group (e.g., carboxylic acid or sulfonic acid group) and at least one alkaline group (e.g., amino group). Acidic groups can be deprotonated using typical bases such as, for example, organic amines or alkali- or alkaline earth hydroxides.

Preferred zwitterionic or amphoteric polymers are:

copolymers formed from alkylacrylamide, alkylaminoalkyl methacrylate, and two or more monomers from acrylic acid and methacrylic acid as well as, if necessary, their esters, especially copolymers from octylacrylamide, acrylic acid, butylaminoethyl methacrylate, methyl methacrylate and hydroxypropyl methacrylate (INCI designation: Octylacrylamide/Acrylate/Butylaminoethyl Methacrylate Copolymer); copolymers, that are formed from at least one of a first type of monomer that possesses quaternary amino groups and at least one of a second type of monomer that possesses acid groups; copolymers from fatty alcohol acrylates, alkylamine oxide methacrylate and at least one monomer selected from acrylic acid and methacrylic acid as well as if necessary acrylic acid esters and methacrylic acid esters, especially copolymers from lauryl acrylate, stearyl acrylate, ethylamine oxide methacrylate and at least one monomer selected from acrylic acid and methacrylic acid as well as if necessary their esters; copolymers from methacryloyl ethyl betaine and at least one monomer selected from methacrylic acid and methacrylic acid esters; copolymers from acrylic acid, methyl acrylate and methacrylamidopropyltrimethylammonium chloride (INCI designation: Polyquaternium-47); copolymers from acrylamidopropyltrimethylammonium chloride and acrylates or copolymers from acrylamide, acrylamidopropyltrimethylammonium chloride, 2-amidopropylacrylamide sulfonate, and dimethylaminopropylamine (INCI designation: Polyquaternium-43); oligomers or polymers, producible from quaternary crotonoylbetaines or quaternary crotonoylbetaine esters.

In one embodiment, the agent according to the invention, as a hair-conditioning or hair-setting additive, contains at least one cationic polymer, i.e., a polymer with cationic or cationizable groups, especially primary, secondary, tertiary, or quaternary amine groups in an amount preferably of from 0.01 to 20 wt. % or of from 0.05 to 10 wt. %, with 0.1 to 5 wt. % being especially preferred. The cationic charge density is preferably 1 to 7 meq/g.

The suitable cationically active polymers are preferably hair-setting or hair-conditioning polymers. Suitable cationic polymers preferably contain quaternary amino groups. Cationic polymers can be homo- or copolymers, where the quaternary nitrogen groups are contained either in the polymer chain or preferably as substituents on one or more of the monomers. The monomers containing ammonium groups can be copolymerized with non-cationic monomers. Suitable cationic monomer are unsaturated compounds that can undergo radical polymerization, which bear at least one cationic group, especially ammonium-substituted vinyl monomers such as, for example, trialkylmethacryloxyalkylammonium, trialkylacryloxyalkylammonium, dialkyldiallylammonium and quaternary vinylammonium monomers with cyclic, cationic nitrogen-containing groups such as pyridinium, imidazolium or quaternary pyrrolidones, e.g., alkylvinylimidazolium, alkylvinylpyridinium, or alkylvinylpyrrolidone salts. The alkyl groups of these monomers are preferably lower alkyl groups such as, for example, C1 to C7 alkyl groups, and especially preferred are C1 to C3 alkyl groups.

The monomers containing ammonium groups can be copolymerized with non-cationic monomers. Suitable comonomers are, for example, acrylamide, methacrylamide, alkyl- and dialkylacrylamide, alkyl- and dialkylmethacrylamide, alkyl acrylate, alkyl methacrylate, vinylcaprolactone, vinylcaprolactam, vinylpyrrolidone, vinyl esters, for example vinyl acetate, vinyl alcohol, propylene glycol or ethylene glycol, wherein the alkyl groups of these monomers are preferably C1 to C7 alkyl groups, and especially preferred are C1 to C3 alkyl groups.

Suitable polymers with quaternary amino groups are, for example, those described in the CTFA Cosmetic Ingredient Dictionary under the designations Polyquaternium such as methylvinylimidazolium chloride/vinylpyrrolidone copolymer (Polyquaternium-16) or quaternized vinylpyrrolidone/dimethylaminoethyl methacrylate copolymer (Polyquaternium-11) as well as quaternary silicone polymers or silicone oligomers such as, for example, silicone polymers with quaternary end groups (Quaternium-80).

Preferred cationic polymers of synthetic origin:

poly(dimethyldiallylammonium chloride); copolymers from acrylamide and dimethyldiallylammonium chloride; quaternary ammonium polymers, formed by the reaction of diethyl sulfate with a copolymer from vinylpyrrolidone and dimethylaminoethyl methacrylate, especially vinylpyrrolidone/dimethylaminoethyl methacrylate methosulfate copolymer (e.g., Gafquat® 755 N, Gafquat® 734); quaternary ammonium polymers from methylvinylimidazolium chloride and vinylpyrrolidone (e.g., LUVIQUAT® HM 550); Polyquaternium-35; Polyquaternium-57; polymers from trimethylammonium ethyl methacrylate chloride; terpolymers from dimethyldiallylammonium chloride, sodium acrylate and acrylamide (e.g., Merquat® Plus 3300); copolymers from vinylpyrrolidone, dimethylaminopropyl methacrylamide and methacryloylaminopropyllauryldimethylammonium chloride; terpolymers from vinylpyrrolidone, dimethylaminoethyl methacrylate and vinylcaprolactam (e.g., Gaffix® VC 713); vinylpyrrolidone/methacrylamidopropyltrimethylammonium chloride copolymers (e.g., Gafquat® HS 100); copolymers from vinylpyrrolidone and dimethylaminoethyl methacrylate; copolymers from vinylpyrrolidone, vinylcaprolactam and dimethylaminopropylacrylamide; poly- or oligoesters formed from at least one first type of monomer, that is selected from hydroxyacids substituted with at least one quaternary ammonium group; dimethylpolysiloxane substituted with quaternary ammonium groups in the terminal positions.

Suitable cationic polymers that are derived from natural polymers are especially cationic derivatives of polysaccharides, for example, cationic derivatives of cellulose, starch or guar. Furthermore, chitosan and chitosan derivatives are also suitable. Cationic polysaccharides are, for example, represented by the general formula

G-O-B-N⁺R^{a}R^{b}R^{c}X—
G is an anhydroglucose residue, for example, starch or cellulose anhydroglucose;
B is a divalent linking group, for example alkylene, oxyalkylene, polyoxyalkylene or hydroxyalkylene;
R^{a}, R^{b}, and R^{c}, independently from one another, are alkyl, aryl, alkylaryl, arylalkyl, alkoxyalkyl, or alkoxyaryl, any of which can have up to 18 C atoms, wherein the total number of C atoms in R^{a}, R^{b}, and R^{c} is preferably a maximum of 20;
X is a conventional counter-anion, for example, a halide, acetate, phosphate, nitrate, or alkyl sulfate, preferably a chloride. Cationic celluloses are, for example, those with the INCI names Polyquaternium-4, Polyquaternium-10, or Polyquaternium-24. A suitable cationic guar derivative has, for example, the INCI designation Guar Hydroxypropyltrimonium Chloride.

Especially preferred cationically-active substances are chitosan, chitosan salts and chitosan derivatives. Chitosans that can be used according to the invention can be fully or partially deacetylated chitins. By way of example, the molecular weight can be distributed over a broad range, from 20,000 to about 5 million g/mol, for example from 30,000 to 70,000 g/mol. However, the molecular weight will preferably lie above 100,000 g/mol, and especially preferred from 200,000 to 700,000 g/mol. The degree of deacetylation is preferably from 10 to 99%, and especially preferably from 60 to 99%. A preferred chitosan salt is chitosonium pyrrolidone carboxylate, e.g., Kytamer® PC with a molecular weight of from about 200,000 to 300,000 g/mol and a degree of deacetylation of from 70 to 85%. Chitosan derivatives that can be considered include quaternized, alkylated or hydroxyalkylated derivatives, e.g., hydroxyethyl, hydroxypropyl or hydroxybutyl chitosan. The chitosans or chitosan derivatives are preferably present in their neutralized or partially neutralized form. The degree of neutralization will be preferably at least 50%, especially preferably between 70 and 100%, as calculated on the basis of the number of free base groups. For the neutralization agent, in principle any cosmetically compatible inorganic or organic acids can be used such as, for example, formic acid, tartaric acid, malic acid, lactic acid, citric acid, pyrrolidone carboxylic acid, hydrochloric acid and others, of which pyrrolidone carboxylic acid is especially preferred.

Preferred cationic polymers derived from natural sources:

cationic cellulose derivatives from hydroxyethyl cellulose and diallyldimethyl ammonium chloride; cationic cellulose deviates from hydroxyethyl cellulose and trimethylammonium-substituted epoxide; chitosan and its salts; hydroxyalkyl chitosans and their salts; alkylhydroxyalkyl chitosans and their salts; N-hydroxyalkylchitosan alkyl ethers.

In one embodiment, the agent according to the present invention contains 0.01 to 15 wt. %, or preferably 0.5 to 10 wt. %, of at least one synthetic or natural nonionic film-forming polymer. Suitable synthetic nonionic polymers are homo- or copolymers consisting of at least one of the following monomers: vinyl lactams such as, for example, vinyl pyrrolidone or vinyl caprolactam; vinyl esters such as, for example, vinyl acetate; vinyl alcohol, vinyl formamide, acrylamides, methacrylamides, alkyl acrylamides, dialkyl acrylamides, alkyl methacrylamides, dialkyl methacryl amides, alkyl acrylates, alkyl methacrylates, alkyl maleimides such as, for example, ethylmaleimide or hydroxyethylmaleimide, and alkylene glycols such as, for example, propylene or ethylene glycol, wherein the alkyl and/or alkylene groups of these monomers are preferably C1 to C7 alkyl groups, or especially preferably C1 to C3 alkyl groups.

Suitable homopolymers are, for example, those of vinylcaprolactam, vinylpyrrolidone or N-vinylformamide. Further suitable synthetic, nonionic polymers are, for example, polyacrylamides, polyethylene glycol/polypropylene glycol copolymers, copolymerides from vinylpyrrolidone and vinyl acetate, terpolymers from vinylpyrrolidone, vinyl acetate, and vinyl propionate, polyacrylamides; polyvinyl alcohols as well as polyethylene glycol/polypropylene glycol copolymers. Suitable natural film-forming polymers are, in particular, those based on saccharide, preferably glucans, e.g., cellulose and derivatives thereof. Suitable derivatives are, in particular, those with alkyl and/or hydroxyalkyl substituents, wherein the alkyl groups can have, for example, 1 to 20, or preferably 1 to 4 C atoms, e.g., hydroxyalkyl cellulose. Preferred nonionic polymers are: polyvinylpyrrolidone, polyvinylcaprolactam, vinylpyrrolidone/vinyl acetate copolymers, polyvinyl alcohol, isobutylene/ethylmaleimide/hydroxyethylmaleimide copolymer; copolymers from vinylpyrrolidone, vinyl acetate, and vinyl propionate.

In one embodiment, the agent according to the present invention contains 0.01 to 20 wt. %, especially preferably 0.05 to 10 wt. %, or very especially preferably 0.1 to 5 wt. % of at least one hair-conditioning additive, selected from A-B block copolymers from alkyl acrylates and alkyl methacrylates; A-B block copolymers from alkyl methacrylates, and acrylonitrile; A-B-A block copolymers from lactide and ethylene oxide; A-B-A block copolymers from caprolacton and ethylene oxide; A-B-C block copolymers from alkylene or alkadiene compounds, styrene and alkyl methacrylates; A-B-C block copolymers from acrylic acid, styrene, and alkyl methacrylates; star-shaped block copolymers; hyper-branched polymers; dendrimers; intrinsically electrically conducting 3,4-polyethylene dioxythiophenes and intrinsically electrically conducting polyanilines.

In one embodiment, the agent according to the invention contains 0.01 to 5 wt. %, or especially preferably 0.05 to 1 wt. %, of at least one preservative. Suitable preservatives are those materials listed with the “Preservatives” function in the International Cosmetic Ingredient Dictionary and Handbook, 10th edition, e.g., phenoxyethanol, benzylparaben, butylparaben, ethylparaben, isobutylparaben, isopropylparaben, methylparaben, propylparaben, iodopropynyl butylcarbamate, methyldibromoglutaronitrile, and DMDM hydantoin.

In a preferred embodiment, the agent according to the invention contains at least one pigment. The pigments can be colored pigments that provide coloring effects to the product mass or the hair, or they can be shine-enhancing pigments that provide shine effects to the product or the hair. The color or shine effects in the hair are preferably temporary, i.e., they remain until the next time the hair is washed and can be removed by washing the hair with typical shampoos. The pigments are not dissolved in the product mass and can be contained in a quantity of from 0.01 to 25 wt. %, with 5 to 15 wt. % being particularly preferred. The preferred particle size is 1 to 200 μm, or particularly 3 to 150 μm, and especially preferably 10 to 100 μm. The pigments are practically insoluble colorants in the application medium and can be inorganic or organic. Inorganic-organic mixed pigments are also possible. Inorganic pigments are preferred. The advantage of inorganic pigments is their extraordinary resistance to light, weather, and temperature. The inorganic pigments can be of natural origin, for example, manufactured from chalk, ocher, umbra, green earth, burnt Terra di Siena, or graphite. The pigments can also be white pigments such as, for example, titanium dioxide or zinc oxide; black pigments such as, for example, iron oxide black; color pigments such as, for example, ultramarine or iron oxide red; shine pigments; metal effect pigments; pearl shine pigments; as well as fluorescence or phosphorescence pigments; wherein it is preferred if at least one pigment is a colored, nonwhite pigment. Metallic oxides, metallic hydroxides, and metallic oxide hydrates, mixed phase pigments, sulfur-containing silicates, metallic sulfides, complex metal cyanides, metallic sulfates, metallic chromates, and metallic molybdates, as well as the metals themselves (bronze pigments) are suitable. Titanium dioxide (CI 77891), black iron oxide (CI 77499), yellow iron oxide (CI 77492), red and brown iron oxide (CI 77491), manganese violet (CI 77742), ultramarine (sodium aluminum sulfosilicates, CI 77007, Pigment Blue 29), chromium oxide hydrate (CI77289), iron blue (ferric ferrocyanide, C177510), and carmine (cochineal) are particularly suitable.

Especially preferred are pearl-shine and color pigments based on mica and/or glimmer, which are coated with a metallic oxide or a metallic oxychloride such as titanium dioxide or bismuth oxychloride as well as, if necessary, other color-providing materials such as iron oxides, iron blue, ultramarine, carmine, etc., and wherein the color can be determined by varying the layer thickness. These types of pigments are sold, for example, under the trade names Rona®, Colorona®, Dichrona®, and Timiron® by Merck, in Germany.

Organic pigments are, for example, the natural pigments sepia, Garcinia gummi-gutta, bone black, Van Dyke brown, indigo, chlorophyll, and other plant pigments. Synthetic organic pigments are, for example, azo-pigments, anthraquinoids, indigoids, and dioxazine, quinacridone, phthalocyanine, isoindolinone, perylene, perinone, metallic complex, alkali blue, and diketopyrrolopyrrol pigments.

In one embodiment, the agent according to the present invention contains 0.01 to 10 wt. %, or especially preferably 0.05 to 5 wt. %, of at least one particle-shaped material. Suitable materials are, for example, materials that are solid and in the form of particles at room temperature (25° C.). Silica, silicates, aluminates, alumina, mica, salts, particularly inorganic metallic salts, metallic oxides, e.g., titanium dioxide, minerals, and polymer particles are somewhat suitable. The particles are present in the agent in an undissolved, preferably steadily dispersed form and can be deposited on the hair in solid form after being applied to the hair and after the solvent has evaporated. A stable dispersion can be obtained by providing the composition with a yield point that is great enough to inhibit any sinking of the solid particles. A sufficient yield point can be obtained by using suitable gel-formers in a suitable quantity. Preferred particle-shaped materials are silica (silica gel, silicium dioxide) and metallic salts, particularly inorganic metallic salts, wherein silica is especially preferred. Metallic salts are, for example, alkaline or alkaline-earth halogenides such as sodium chloride or potassium chloride; and alkaline or alkaline earth sulfates such as sodium sulfate or magnesium sulfate.

An additional embodiment relates to an agent for permanently restructuring hair. It contains at least one reducing agent, particularly a keratin-reducing mercapto compound preferably in a quantity of from 0.5 to 15 wt. %. The permanent wave agent is preferably present as an aqueous, alkaline (pH=5 to 10) preparation, which contains e.g., cysteine, cysteamine, N-acetyl-L-cysteine, mercapto carboxylic acids such as, for example, mercaptoacetic acid or thiolactic acid, or salts of mercapto carboxylic acids such as, for example, ammonium and guanidine salts of mercaptoacetic acid or thiolactic acid as a keratin-reducing mercapto compound. The required alkalinity is obtained by adding ammonia, organic amines, ammonium and alkali carbonates, or bicarbonates. Neutral or acidic (pH=4.5 to 7) hair restructuring agents that have an effective content of sulfites or mercaptocarboxylic acid esters in an aqueous medium can also be considered. In the first case, preferably sodium or ammonium sulfite or the salt of sulfuric acid with an organic amine such as, for example, monoethanolamine and guanidine, can be used in a concentration of approximately 2 to 12 wt. % (calculated as SO₂). In the latter case, mercaptoacetic acid mono glycol esters or glycerin esters are particularly used in a concentration of approximately 5 to 50 wt. % (corresponding to a content of 2 to 16 wt. % mercaptoacetic acid). The agent according to the invention for permanent restructuring of hair can also contain a mixture of the aforementioned keratin-reducing compounds. For the oxidative after-treatment, a fixing agent according to the invention containing at least one oxidizing agent can be used. Examples of oxidizing agents that can be used in one of these types of fixing agents are sodium and potassium bromate, sodium perborate, urea peroxide, and hydrogen peroxide. The concentration of oxidizing agent can be approximately 0.5 to 10 wt. %. Both the agent according to the invention for permanent hair restructuring as well as the fixing agent according to the invention can be present in the form of an emulsion or in thickened form on an aqueous basis, particularly as a cream, gel, or paste.

The composition to be used according to the invention can further contain any additive components that are conventional for hair treatment agents, for example perfume oils; opacifying agents such as, for example, ethylene glycol distearate, styrene/PVP copolymers or polystyrenes; humectants; shine providers; product dyes; antioxidants; each preferably in quantities of from 0.01 to 10 wt. %, wherein the total quantity preferably does not exceed 10 wt. %.

The products according to the invention are characterized by simple and precise applicability, good, economical dispensability, good and even distributability, a consistency that is perceived as being more pleasant as compared to conventional sun creams, as well as a pleasant, non-sticky feel on the skin and hair.

A particular embodiment of the invention relates to a hair-conditioning agent. Hair-conditioning agents are, for example, conditioners, treatments, hair-repair products, rinses, and the like. The hair-conditioning agent according to the present invention can, after application to the dry, damp, or wet hair, either remain in the hair or it can be rinsed out after a suitable action period. The action periods depend on the type of hair. As a general rule, action periods of between 0.5 and 30 minutes, particularly 0.5 and 10 minutes, and preferably between 1 and 5 minutes can be assumed.

The object of the invention is also a method for hair or skin treatment, wherein
a product release system according to the invention is provided,
using the product release system, the composition contained therein is sprayed onto the hair or onto the skin, and
the composition that is sprayed on is either rinsed out of the hair after an action period or, preferably, it is left in the hair or on the skin.

Instead of being sprayed directly onto the hair or the skin, the product, particularly if it is in the form of a snow-like substance, flakes, or foam, can also be placed in the hands or on an application device such as, for example, a comb or a brush, and then distributed into the hair.

The products according to the invention are characterized, constrained by their special application with the special aerosol spray system to be used according to the invention, by a high level of UV protective effect on the hair or on the skin. The advantages with use are characterized by a comfortable application, improved distributability, more economical dispensing, a more pleasant consistency as perceived by the user, and a perceptively more pleasant feel on the skin, with UV protection results that are the same as or better than conventional products. The extremely fine spray behavior of the capillary system provides the user with a very even distribution of the product on the hair or on the skin. This leads to improved UV protection results and less consumption, because the product does not have to be distributed in the hands. An additional advantage of the products according to the present invention is that differing spray properties can be precisely adjusted by simply varying the propellant, the propellant composition, or the propellant pressure; these spray properties were not previously possible for the underlying active ingredient compositions. The spray properties include everything from a fine aerosol atomized spray and snow-like drops to flakes of spray and spray foam.

The following examples should serve to illustrate further the object of the present invention.

### EXAMPLES

In the following examples, the individual active ingredient compositions were filled, along with the individually indicated propellants, into a pressure-resistant aerosol can and equipped with a capillary spray system, as can be obtained, for example, under the trade name TRUSPRAY® from Boehringer Ingelheim microParts GmbH.

### Examples 1 through 3

### Sun-Protective Agent for the Skin (Gel Cream)

Active ingredient composition:

| | 1 | 2 | 3 |
|---|---|---|---|
| Octyldodecanol | 10.00 g | 10.00 g | 10.00 g |
| HYDROGENATED COCO-GLYCERIDES | 1.00 g | 1.00 g | 1.00 g |
| Glycerol | 5.00 g | 5.00 g | 5.00 g |
| ACRYLATES/C10-30 ALKYL ACRYLATE | 0.60 g | 0.60 g | 0.60 g |
| CROSSPOLYMER (Carbopol ® 1382) | | | |
| Sodium hydroxide | 0.20 g | 0.20 g | 0.20 g |
| BUTYL | 0.50 g | 0.25 g | 0.40 g |
| METHOXYDIBENZOYLMETHANE | | | |
| METHOXY CINNAMIC ACID | 3.00 g | | 7.50 g |
| OCTYLESTER | | | |
| OCTOCRYLENE | | 5.00 g | 7.50 g |
| METHYLBENZYLIDENE CAMPHOR | | 1.00 g | 3.00 g |
| PHENYLBENZIMIDAZOLE SULFONIC | 3.00 g | | |
| ACID | | | |
| C12-15 ALKYL BENZOATE | | 2.00 g | 1.88 g |
| Perfume | 0.30 g | 0.30 g | 0.30 g |
| Phenoxyethanol | 0.50 g | 0.50 g | 0.50 g |
| Water | balance | balance | balance |
| | to 100 g | to 100 g | to 100 g |

Filling of compositions 1 through 3 with propellant:

| | A | B | C | D | E |
|---|---|---|---|---|---|
| Active ingredient | 50 g | 50 g | 50 g | 60 g | 70 g |
| Propane/butane 4.8 bar | 50 g | | | 40 g | 30 g |
| Propane/butane 2.7 bar | | 50 g | | | |
| DME | | | 50 g | | |

Spray behavior:

1A: Very fine, dry aerosol spray

1B: Fine, wet aerosol spray

1C: Snow-like

1D: Fine, dry aerosol spray

1E: Aerosol spray

### Examples 4 through 6

### Sun-Protective Agent for the Skin (Water-in-Oil Emulsion)

| Raw ingredients | 4 | 5 | 6 |
|---|---|---|---|
| CAPRYLIC/CAPRIC TRIGLYCERIDE | 8.00 g | 8.00 g | 8.00 g |
| Cetearyl isononanoate | 6.00 g | 6.00 g | 6.00 g |
| Carnauba wax | 0.50 g | 0.50 g | 0.50 g |
| Beeswax | 0.50 g | 0.50 g | 0.50 g |
| C12-15 ALKYL BENZOATE | 7.00 g | 7.00 g | 7.00 g |
| Glyceryl oleates | 6.00 g | 6.00 g | 6.00 g |
| PEG-7 HYDROGENATED CASTOR OIL | 4.00 g | 4.00 g | 4.00 g |
| Glycerol | 5.00 g | 5.00 g | 5.00 g |
| PEG-30 DIPOLYHYDROXYSTEARATE | 4.00 g | 4.00 g | 4.00 g |
| Zinc oxide | 4.00 g | 6.00 g | 4.00 g |
| Zinc sulfate-7-hydrate | 1.00 g | 1.00 g | 0.50 g |
| Titanium dioxide | | 2.00 g | 4.00 g |
| ETHYLHEXYL TRIAZONE | 4.00 g | | 6.00 g |
| PHENYLBENZIMIDAZOLE SULFONIC | 8.00 g | 5.60 g | |
| ACID | | | |
| BUTYL | | 5.00 g | 3.00 g |
| METHOXYDIBENZOYLMETHANE | | | |
| METHOXY CINNAMIC ACID | 6.00 g | | 3.00 g |
| OCTYLESTER | | | |
| OCTOCRYLENE | 5.00 g | | |
| METHYLBENZYLIDENE CAMPHOR | | 4.00 g | 3.50 g |
| Methylparabens | 0.20 g | 0.20 g | 0.20 g |
| Perfume | 0.30 g | 0.30 g | 0.30 g |
| Dekaben LMB | 0.50 g | 0.50 g | 0.50 g |
| Water | balance | balance | balance |
| | to 100 g | to 100 g | to 100 g |

### Consistency: Highly viscous, fatty creams

Filling of active ingredient compositions 4 through 6 with propellant:

| | A | B | C | D | E |
|---|---|---|---|---|---|
| Active ingredient | 50 g | 50 g | 50 g | 60 g | 70 g |
| Propane/butane 4.8 bar | 50 g | | | 40 g | 30 g |
| Propane/butane 2.7 bar | | 50 g | | | |
| DME | | | 50 g | | |

Spray behavior:

4A: Very fine aerosol spray

4B: Fine, wet aerosol spray

4C: Snow-like

4D: Fine aerosol spray

4E: Aerosol spray

The applicability of aerosol 4D was compared to that of propellant-free cream 4 by a panel consisting of 10 people.

Test question: How would you rate the following criteria with the aerosol as compared to the cream?
Consistency of the mass
Distributability
Ease of dispensing
Feel on the skin/scalp

Assessment scale:

+3 Much better

+2 Better

+1 Slightly better

0 No difference

−1 Slightly worse

−2 Worse

−3 Significantly worse

The results are compiled in the following table:

| | 4D as compared to 4 | | | | | | | |
|---|---|---|---|---|---|---|---|---|
| Criterion | +3 | +2 | +1 | 0 | −1 | −2 | −3 | Ø |
| Consistency of the mass | 5 | 2 | 2 | 1 | — | — | — | +2.1 |
| Distributability | 4 | 4 | 1 | 1 | — | — | — | +2.1 |
| Ease of dispensing | 1 | 3 | 4 | 1 | 1 | — | — | +1.3 |
| Feel on the skin/scalp | 5 | 2 | 1 | 2 | — | — | — | +2.0 |

Aerosol application 4D was rated as being better than the conventional cream in the criteria examined in the test.

### Examples 7 through 8

### Hair Treatment Agent with UV Filter (Rinse-Off Hair-Repair Product)

| Raw ingredients | 7 | 8 |
|---|---|---|
| Cetyl alcohol | 8.00 g | 8.00 g |
| Liquid paraffin | 3.00 g | 3.00 g |
| Isopropyl myristate | 2.00 g | 2.00 g |
| Cetrimonium chlorides | 1.50 g | 1.50 g |
| Glyceryl oleates | 0.70 g | 0.70 g |
| Coco-glucosides | 0.70 g | 0.70 g |
| METHOXY CINNAMIC ACID OCTYLESTER | 0.50 g | 0.75 g |
| Methylbenzylidene camphor | 2.50 g | |
| PHENYLBENZIMIDAZOLE SULFONIC ACID | | 4.00 g |
| Perfume | 0.30 g | 0.30 g |
| Methylparaben | 0.20 g | 0.20 g |
| Water | balance to | balance to |
| | 100 g | 100 g |

### Consistency: Highly viscous rinse-off hair-repair product

Filling of active ingredient compositions 7 through 8 with propellant:

| | A | B | C | D | E |
|---|---|---|---|---|---|
| Active ingredient | 50 g | 50 g | 50 g | 60 g | 70 g |
| Propane/butane 4.8 bar | 50 g | | | 40 g | 30 g |
| Propane/butane 2.7 bar | | 50 g | | | |
| DME | | | 50 g | | |

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A product release system to atomize a cosmetic composition comprising a pressure-resistant packaging, a spray head comprising a capillary, and a propellant-containing cosmetic composition, wherein the atomization is done using said capillary and said composition comprises at least one UV filter.

2. A product release system according to claim 1, wherein said capillary has a diameter of 0.1 to 1 mm and a length of 5 to 100 mm.

3. A product release system according to claim 1, wherein the spray rate is 0.01 to 5 g/s.

4. A product release system according to claim 1, wherein the propellant is selected from the group consisting of propane, butane, dimethyl ether, fluorinated hydrocarbons, and mixtures thereof.

5. A product release system according to claim 1, wherein the UV filter is selected from the group consisting of materials based on 3-benzylidene camphor, 4-aminobenzoic acid, cinnamic acid esters, salicylic acid, benzophenone, 2-phenylbenzimidazole, and dibenzoylmethane.

6. A product release system according to claim 1, wherein the UV filter is selected from the group consisting of 4-tert-butyl-4′-methoxydibenzoylmethane, 4-methoxy cinnamic acid-2-ethylhexyl ester, methyl methoxy cinnamate, 2-phenyl-5-sulfobenzimidazole, zinc oxide, titanium dioxide, ethylhexyl triazone, 2-hydroxy-4-methoxy benzophenone-5-sulfonic acid, 3-(4-methylbenzylidene)-dl-camphor, polyethoxylated p-aminobenzoates, and 2-ethylhexyl-2-cyano-3,3-diphenyl acrylate.

7. A product release system according to claim 1, wherein the composition is a gel, wax, or emulsion.

8. A product release system according to claim 7, wherein the wherein the composition is a gel comprising at least one thickener or gel-former in a quantity of from 0.01 to 20 wt. %.

9. A product release system according to claim 8, wherein the thickener or gel-former is a thickening polymer, selected from the group consisting of copolymers which are made from at least one first type of monomer and from at least one second type of monomer, wherein said first type of monomer is selected from the group consisting of acrylic acid and methacrylic acid and wherein said second type of monomer is selected from the group consisting of ethoxylated fatty alcohol acrylic acid esters, crosslinked polyacrylic acid, and crosslinked copolymers which are made from at least one first type of monomer, and from at least one second type of monomer, wherein said first type of monomer is selected from the group consisting of acrylic acid and methacrylic acid and wherein said second type of monomer is selected from the group consisting of C10 to C30 alcohol acrylic acid esters, copolymers made from at least one first type of monomer and from at least one second type of monomer, wherein said first type of monomer is selected from the group consisting of acrylic acid and methacrylic acid and wherein said at least one second type of monomer is selected from the group consisting of esters of itaconic acid and ethoxylated fatty alcohol, copolymers from at least one first type of monomer, from at least one second type of monomer and from at least one third type of monomer, wherein said first type of monomer is selected from the group consisting of acrylic acid and methacrylic acid and wherein said at least one second type of monomer is an ester of itaconic acid and ethoxylated C10 to C30 alcohol and wherein said third type of monomer is selected from the group consisting of C1 to C4 aminoalkyl acrylates and copolymers from two or more monomers selected from the group consisting of acrylic acid, methacrylic acid, acrylic acid esters, and methacrylic acid esters, copolymers of vinyl pyrrolidone and ammonium acryloyl dimethyltaurate, copolymers of ammonium acryloyl dimethyltaurate and at least one second monomer, wherein said second monomer is an ester of methacrylic acid and ethoxylated fatty alcohol, hydroxyethyl cellulose, hydroxypropyl cellulose, hydroxypropyl guar, glyceryl polyacrylate, glycerylpoly methacrylate, copolymers from at least one first type of monomer and styrene, wherein said first type of monomer is selected from the group consisting of C2 alkylene, C3 alkylene and C4 alkylene, polyurethanes, hydroxypropyl starch phosphate, polyacrylamide, copolymers of maleic acid anhydride and methyl vinyl ether, wherein said copolymers of maleic acid are crosslinked with decadiene, locust bean gum, guar gum, xanthan, dehydroxanthan, carrageenan, karaya gum, hydrolyzed corn starch, copolymers from polyethylene oxide, fatty alcohols, and saturated methylene diphenyl diisocyanate.

10. A product release system according to claim 7, wherein the composition is a waxy composition comprising at least one wax that is solid at 25° C. in a quantity of from 10 to 80 wt. % of the composition.

11. A product release system according to claim 10, wherein said wax is selected from the group consisting of paraffin waxes, polyolefin waxes, wool wax, wool wax alcohols, candelilla wax, olive wax, carnauba wax, Japan wax, apple wax, hydrogenated fats, fatty acid esters, fatty acid glycerides, fatty acid triglycerides, polyethylene glycol waxes and mixtures thereof.

12. A product release system according to claim 7, wherein the composition is an emulsion-type composition selected from the group consisting of water-in-oil emulsions, oil-in-water emulsions and microemulsions, and wherein the composition comprises at least one emulsifier in a quantity of from 0.1 to 30 wt. %, and at least one oil in a quantity of from 1 to 20 wt. %, and water.

13. A product release system according to claim 12, wherein the emulsifier is selected from the group consisting of addition products of 2 to 30 mol ethylene oxide to C8 to C22 fatty alcohols addition products of 1 to 5 mol propylene oxide to C8 to C22 fatty alcohols, addition products of 2 to 30 mol ethylene oxide and 1 to 5 mol propylene oxide to C8 to C22 fatty alcohols, addition products of 2 to 30 mol ethylene oxide to C12 to C22 fatty acids, addition products of 1 to 5 mol propylene oxide to C12 to C22 fatty acids, addition products of 2 to 30 mol ethylene oxide and 1 to 5 mol propylene oxide to C12 to C22 fatty acids, addition products of 2 to 30 mol ethylene oxide alkyl phenols with 8 to 15 C atoms in the alkyl group, addition products of 1 to 5 mol propylene oxide to alkyl phenols with 8 to 15 C atoms in the alkyl group, addition products of 2 to 30 mol ethylene oxide and 1 to 5 mol propylene oxide alkyl phenols with 8 to 15 C atoms in the alkyl group, C12 to C22 fatty acid monoesters of addition products of 1 to 30 mol ethylene oxide to glycerol, C12 to C22 fatty acid diesters of addition products of 1 to 30 mol ethylene oxide to glycerol, addition products of 5 to 60 mol ethylene oxide to castor oil, addition products of 5 to 60 mol ethylene oxide to hydrogenated castor oil, mono-, di-, or triesters of phosphoric acid with addition products of 2 to 30 mol ethylene oxide to C8 to C22 fatty alcohols, esters of saccharose with one or two C8 to C22 fatty acids, esters of sorbitan and one, two, or three C8 to C22 fatty acids and having a degree of ethoxylation of from 4 to 20, polyglyceryl fatty acid esters of one, two, or more C8 to C22 fatty acids with polyglycerol of 2 to 20 glyceryl units, and alkylglycosides.

14. A product release system according to claim 1, wherein said composition comprises at least one additional active ingredient selected from the group consisting of skin-conditioning materials, hair-conditioning materials, hair-setting materials, silicone compounds, preservatives, pigments, particle-shaped materials, and mixtures thereof.

15. A product release system according to claim 14, comprising said active ingredients in a quantity of from 0.01 to 20 wt. % of said composition.

16. A product release system according to claims 14, further comprising at least one silicone compound, which is selected from the group consisting of cyclic dimethylsiloxanes, linear polydimethylsiloxanes, block polymers from polydimethylsiloxane and at least one block selected from the group consisting of polyethylene oxide and polypropylene oxide, polydimethylsiloxanes with terminal or lateral residues selected from the group consisting of polyethylene oxide and polypropylene oxide, polydimethylsiloxanes with terminal hydroxyl groups, phenyl-substituted polydimethylsiloxanes, silicone emulsions, silicone elastomers, silicone waxes, silicone gums, amino-substituted silicones, and silicones substituted with one or more quaternary ammonium groups.

17. A product release system according to claim 14, further comprising an active ingredient, which is selected from the group consisting of betaine, panthenol, panthenyl ethyl ether, sorbitol, protein hydrolysates, plant extracts, A-B block copolymers from alkyl acrylates and alkyl methacrylates, A-B block copolymers from alkyl methacrylates and acrylonitrile, A-B-A block copolymers from lactide and ethylene oxide, A-B-A block copolymers from caprolacton and rethylene oxide, A-B-C block copolymers from alkylene or alkadiene compounds, styrene and alkyl methacrylates, A-B-C block copolymers from acrylic acid, styrene, and alkyl methacrylates, star-shaped block copolymers, hyper-branched polymers, dendrimers, intrinsically electrically conducting 3,4-polyethylene dioxythiophenes, and intrinsically electrically conducting polyanilines.

18. A product release system according to claim 14, wherein said active ingredient is selected from the group consisting of hair-conditioning polymers, hair-setting polymers, silicone compounds, preservatives, pigments, direct-penetrating hair dyes, particle-shaped materials, oxidizing agents, reducing agents, oxidative hair dye precursor products and mixtures thereof.

19. A product release system according to claim 14, wherein said active ingredient is selected from the group consisting of antioxidants, natural ingredients, anti-wrinkle ingredients, self-tanning substances, repellents, moisturizers, fillers that improve the feel of the skin, and mixtures thereof.

20. A product release system according to claim 14, further comprising at least one pigment, which is selected from the group consisting of titanium dioxide, black iron oxide, yellow iron oxide, red and brown iron oxide, manganese violet, ultramarine, chromium oxide hydrate, iron blue, bismuth oxichloride, carmine (cochineal), mica based pearl shine pigments, mica based color pigments, wherein said mica based pearl shine pigments and said mica based color pigments are coated with a coating selected from the group consisting of metallic oxides and metallic oxychlorides and wherein the different colors of the mica based color pigments are produced by different layer thicknesses.

21. A product release system according to claim 14, further comprising at least one particle-shaped material, which is selected from the group consisting of silica, silicates, aluminates, alumina, mica, insoluble metallic salts, metallic oxides, minerals, insoluble polymer particles and mixtures thereof.

22. A product release system according to claim 1, wherein the composition is in the form of an oil-in-water emulsion, a water-in-oil emulsion, or a microemulsion and comprises;
(a) from 1 to 20 wt. % of at least one oil or fatty alcohol,
(b) from 0.1 to 30 wt. % of at least one emulsifier, which is selected from the group consisting of cationic surfactants, anionic surfactants, zwitterionic surfactants, and nonionic surfactants, and
(c) from 0.1 to 30 wt. % of at least one UV filter.
